(19) United States
(12) Patent Application Publication
(10) Pub. No.: US 2021/0267385 A1
(43) Pub. Date: Sep. 2, 2021

(54) ROCKING BASSINET

(71) Applicant: The Boppy Company, LLC, Golden, CO (US)

(72) Inventors: Sharon Gillett, Ortig, WA (US); Julia Larrabee, Boulder, CO (US)

(21) Appl. No.: 17/178,904

(22) Filed: Feb. 18, 2021

Related U.S. Application Data
(60) Provisional application No. 62/982,264, filed on Feb. 27, 2020.

(51) Int. Cl. A47D 9/04; A47D 15/00; A61M 21/02

(52) U.S. Cl. CPC A47D 9/04; A61M 2021/0022; A61M 21/02; A47D 15/00

(57) ABSTRACT

A bassinet for supporting an infant and providing clear lines of sight for a caregiver through the walls of the bassinet basket. The bassinet basket rocks about a single axis with respect to the frame of the bassinet. The bassinet basket is formed substantially of a mesh material that allows visual observation of the interior of the bassinet basket. The bassinet basket includes an infant insert that suspends a padded region between a top and bottom of the bassinet basket and forms a storage compartment under the infant insert. The frame of the bassinet is height adjustable. The bassinet also includes a control panel for various electrical features and elements of the bassinet.

### CROSS REFERENCE TO RELATED APPLICATIONS

This application claims priority to U.S. Provisional Patent Application No. 62/982,264, filed Feb. 27, 2020, the disclosure of which is incorporated by reference herein in its entirety.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Bassinets or cradles are well known and are generally used to provide a secure place for sleeping infants. Bassinets may take on numerous different configurations. Bassinets are typically small and are used to keep the infant close to the parents while the infant is sleeping. Typically, bassinets are elevated above the ground and include a frame to form a flat horizontal space enclosed with sidewalls. The flat space is large enough to accommodate an infant when the infant is lying down for sleeping. Bassinets also generally may include a canopy to shade the infant from excess light. Some bassinets may be capable of being rocked by hand, and others may be provided with a mechanism to automatically rock the bassinet.

### BRIEF SUMMARY

The embodiments discussed below include a rocking bassinet that enables viewing of an infant within the bassinet from nearly any angle. The rocking bassinet includes a height-adjustable frame as well as a removable bassinet basket. The removable bassinet basket may rotate within a limited range of motion with respect to the height-adjustable frame. The removable bassinet may include an infant insert suspended from the upper perimeter of the bassinet basket to support a sleeping infant.

In some embodiments, the bassinet for an infant may include a rocking and removable basket, and a height-adjustable frame. The bassinet is constructed in a manner that provides 360° sightlines into the basket to view an infant placed therein. The bassinet may include a height-adjustable frame or base including lockable wheels, a removable rocking basket, and a removable control panel. The height-adjustable frame may include a single actuation point which releases locking members on each the legs of the frame simultaneously to provide ease of height adjustment. The removable rocking basket may connect to the height-adjustable frame such that the basket can pivot about a single axis when connected to the height-adjustable frame. The connection between the basket and the height-adjustable frame may include rotation limit features to limit the range of rotation of the basket about the single axis. The connection may also securely lock the basket to the height-adjustable frame such that it can only be removed by actuating a locking element. The basket may connect to the height-adjustable frame at or near an upper perimeter of the basket and includes wall materials that are transparent or porous through which a caregiver can view the interior of the basket. Additionally, the bottom of the basket may be formed of the same transparent or porous material to provide full visibility to a caregiver from any angle and to provide breathability. The removable control panel may include features such as controllable lights, sounds, displays, vibration settings, and other such settings selectable by a caretaker.

In other embodiments, a bassinet includes a bassinet basket including mesh side panel and a bottom panel, the mesh side panel and bottom panel allowing air movement through the mesh side and bottom panels and allowing visual inspection of an interior of the bassinet basket from a variety of surrounding positions. The bassinet also includes an infant insert removably coupleable to an upper rim of the bassinet basket, the infant insert configured to support an infant above the bottom panel of the bassinet basket and forming an open compartment between the bassinet basket and the infant insert. The bassinet also includes a frame having a support portion for resting against a supporting surface and supports.

In some embodiments, the bassinet may include a storage unit underneath the removable basket or a storage pouch that can be attached to the frame, as well as a removable infant insert that couples to the rocking basket. The storage unit may include a space underneath the infant insert within the basket. In some examples, a storage unit may couple to the height-adjustable frame external to the basket.

### BRIEF DESCRIPTION OF THE DRAWINGS

FIG. 1 depicts a perspective view of a rocking bassinet, according to at least some embodiments.

FIG. 2 depicts a perspective view of a rocking bassinet with a height-adjustable frame and an infant insert, according to at least some embodiments.

FIG. 3 depicts a perspective view of the rocking bassinet of FIG. 2 with the infant insert removed, according to at least some embodiments.

FIG. 4 depicts a perspective view of the frame of the rocking bassinet of FIG. 1, according to at least some embodiments.

FIG. 5 depicts a perspective view of the height-adjustable frame of the rocking bassinet of FIG. 2, according to at least some embodiments.

FIG. 6 depicts a perspective view of the rocking bassinet of FIG. 2, according to at least some embodiments.

FIG. 7 depicts a partial view of the rocking bassinet of FIG. 1 including an infant insert connected to a frame, according to at least some embodiments.

FIG. 8 depicts a perspective view of the basket of the rocking bassinet of FIG. 1, according to at least some embodiments.

FIG. 9 depicts a side view of the basket of FIG. 8, according to at least some embodiments.

FIG. 10 depicts a basket including the infant insert of the rocking bassinet of FIG. 2, according to at least some embodiments.

FIG. 11 depicts a perspective view of the basket of the rocking bassinet of FIG. 10, according to at least some embodiments.

FIG. 12 depicts an infant insert for connecting to a basket of a bassinet, according to at least some embodiments.

FIG. 13 depicts a perspective view of a control panel for a bassinet, according to at least some embodiments.

FIG. 14 depicts a bottom view of the control panel of FIG. 13, according to at least some embodiments.

FIG. 15 depicts a rocking bassinet having a storage caddy attached to the frame, according to at least some embodiments.

FIG. 16. depicts a top view of the rocking bassinet of FIG. 1, according to at least some embodiments.

### DETAILED DESCRIPTION OF THE INVENTION

One or more specific embodiments of the present invention will be described below. These embodiments are only exemplary of the present invention. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The discussion below describes embodiments of a bassinet for an infant that provides a rocking removable basket, a height-adjustable frame, and 360° sightlines into the basket to view an infant placed therein. The term infant is understood to mean a child up to one year of age and weighing up to approximately twenty-five pounds, although in some cases, the bassinet may be used for an infant of up to around five to six months of age or when they start to roll over or push up on their knees. The bassinet may also include a height-adjustable frame or base including lockable wheels, a removable rocking basket, and a removable control panel. The height-adjustable frame may include a single actuation point which releases locking members on each the legs of the frame simultaneously to provide ease of height adjustment. The removable rocking basket connects to the height-adjustable frame such that the basket can pivot about a single axis when connected to the height-adjustable frame. The connection between the basket and the height-adjustable frame may include rotation limit features to limit the range of rotation of the basket about the axis. The connection also securely locks the basket to the height-adjustable frame and can only be removed by actuating a locking element. The basket connects to the height-adjustable frame at or near an upper perimeter of the basket and includes wall materials that are transparent or porous through which a caregiver can view the interior of the basket. Additionally, the bottom of the basket may be formed of the same transparent or porous material to provide full visibility to a caregiver from any angle and to provide breathability. The removable control panel may include features such as controllable lights, sounds, displays, vibration settings, and other such settings selectable by a caretaker.

In some embodiments, the bassinet may include a storage unit underneath the removable basket or a storage pouch that can be attached to the frame, as well as a removable infant insert that couples to the rocking basket. The storage unit may include a space underneath the infant insert within the basket. In some examples, a storage unit may couple to the height-adjustable frame external to the basket.

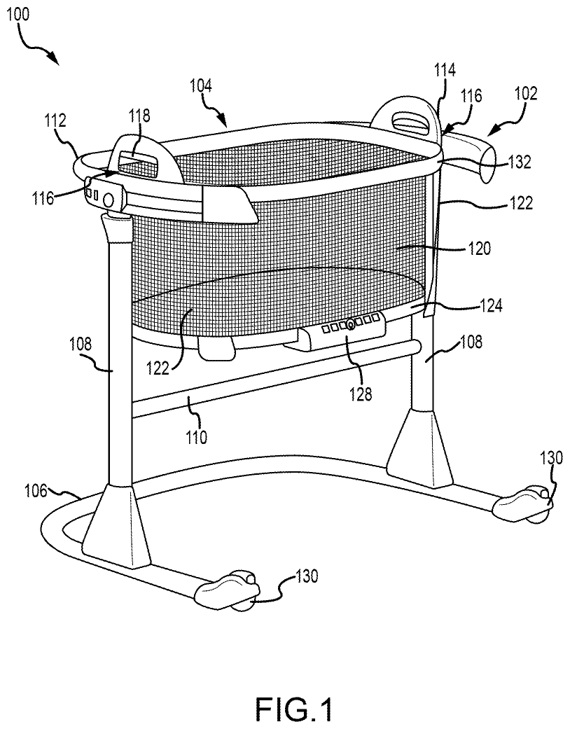

FIG. 1 depicts a perspective view of a bassinet 100, according to at least some embodiments. The bassinet 100 includes a frame 102 as well as a basket 104. The bassinet 100 is configured so that the basket 104 can be removed from the frame 102. Additionally, as described below, the basket 104 may rock or pivot with respect to the frame 102, such as to provide a swaying motion for an infant lying therein. The bassinet 100 may be height-adjustable and may also provide a variety of storage solutions. Bassinet 100 also provides a caregiver the ability to view an infant from essentially any viewpoint as described below.

The frame 102 has a “U”-shaped foot 106 to which a pair of wheels 130 are connected. The wheels 130 are connected to the ends of the “U”-shaped foot 106 such that the bassinet 100 rests on a supporting surface on the “U”-shaped foot 106 as well as the wheels 130. The wheels 130 may be locked to prevent movement of the bassinet 100 while in use. In some examples, the wheels 130 may be actuated to selectively contact the supporting surface. For example, pressing a lever, button, or latch may cause the wheels 130 to descend or extend and contact the supporting surface, thereby bearing at least part of the weight of the bassinet 100. The wheels 130 and the “U”-shaped foot 106 may enable the bassinet 100 to be moved with ease by a caregiver when the wheels 130 are unlocked. In some examples, the bassinet 100 may be tilted with respect to the supporting surface such that the wheels 130 are the only contact point of the bassinet 100 with the supporting surface. This may allow a caregiver to easily move the bassinet to any desired location or position. Though only two wheels 130 are shown in FIG. 1, in some examples there may be fewer or greater numbers of wheels 130, such as wheels arranged around different positions of the “U”-shaped foot 106.

Extending upwards from the “U”-shaped foot 106 are two supports 108. The supports 108 extend perpendicular or substantially perpendicular to the supporting surface or a plane of the “U”-shaped foot 106. In some examples, the supports 108 may extend from one edge, such as a center portion, of the “U”-shaped foot 106 and the angled or cantilevered to maintain the basket 104 in a central position of the bassinet. There may be more or fewer than two supports 108 in some embodiments. For example, there may be one, three, four, or more supports 108 extending from the “U”-shaped foot 106. The supports 108 may be height-adjustable, either manually or automatically. One example of a manual height adjustment includes a feature depressed by a user (such as a latch) which releases a securing pin and allows the supports 108 to be extended or shortened before re-securing the pin by releasing the latch and allowing the pin to pass through a port in the wall of supports 108. In such examples, the supports 108 may include two or more segments arranged axially and concentrically with respect to each other such that when moved relative to each other along the axis the support 108 is extended or shortened. In an example of an automatic height adjustment, each support 108 may be outfitted with one or more linear actuators. The linear actuators may advance segments or sections of the supports 108 to extend or shorten the length of the supports. In this example, the linear actuators may be controlled through a computing system or other electronic controller and activated by a switch depressed by a caregiver.

The supports 108 are joined by a crossbar 110 which provides strength, rigidity, and support between the supports 108. The crossbar 110 may be a simple beam that extends between the supports 108. The crossbar 110 may also include a height adjustment latch as described below to release the supports 108 for extension or shortening.

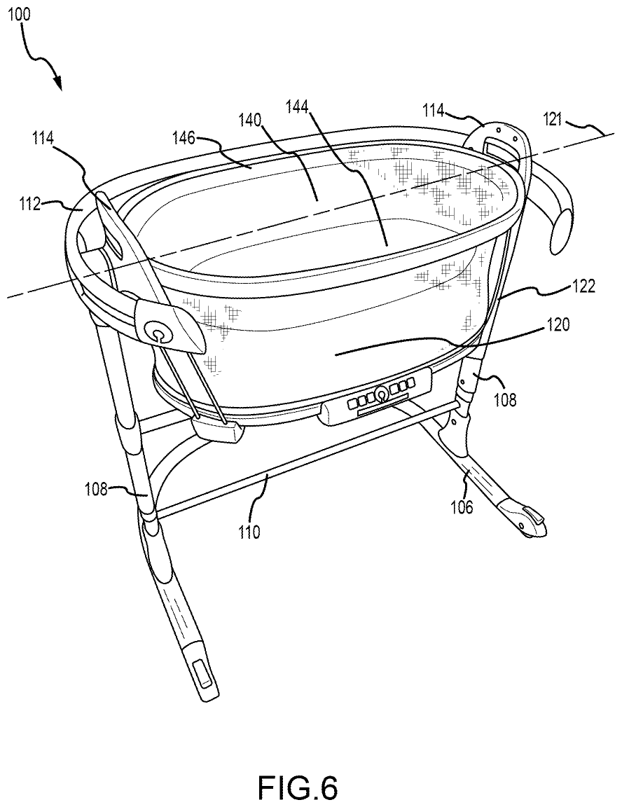

At the upper end of the supports 108, an upper rail 112 provides a connection to the basket 104. The upper rail 112 is shown having a “C”-shape, though other configurations may be used. For example, the upper rail 112 may be formed of multiple segments, such as an upper rail 112 at each end of the basket 104. At opposite ends of the upper rail 112 there may be basket connections 116. The basket connections 116 enable the basket 104 to be removed from the frame 102. The basket connections 116 also enable rocking of the basket 104 around an axis which passes through both of the basket connections 116. The axis is shown as axis 121 of FIG. 6. The basket connections 116 also limit the degree of rotation of the basket 104 about the axis.

For example, the basket connections 116 may prevent the basket 104 from rotating beyond ninety degrees with respect to the supporting surface from the neutral position. In some embodiments, the rotation may be limited to less than seventy-five degrees, sixty degrees, fifty degrees, forty degrees, or any other suitable rotation range.

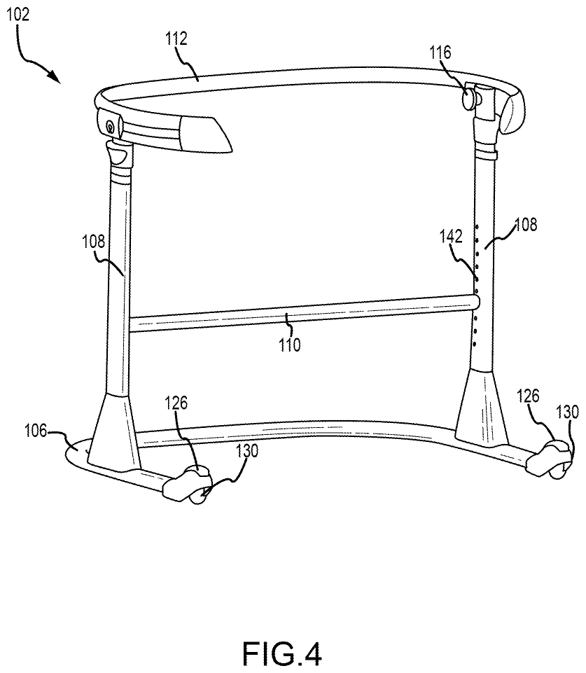

The basket 104 is described in further detail below, with respect to FIGS. 4-7. The basket 104 includes handles 114 at opposite ends of the basket 104 with release latches 118 inside of each handle 114. The handles 114 may enable a caregiver to lift or carry the basket 104. The release latches 118 actuate a latching mechanism that connects to the basket connections 116 to secure the basket 104 to the frame 102. The latching mechanism still allows the rotation described above, even when the basket 104 is latched to the frame 102.

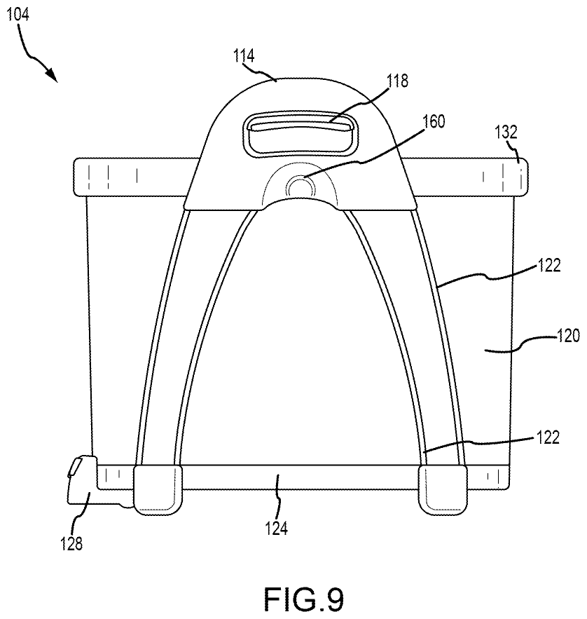

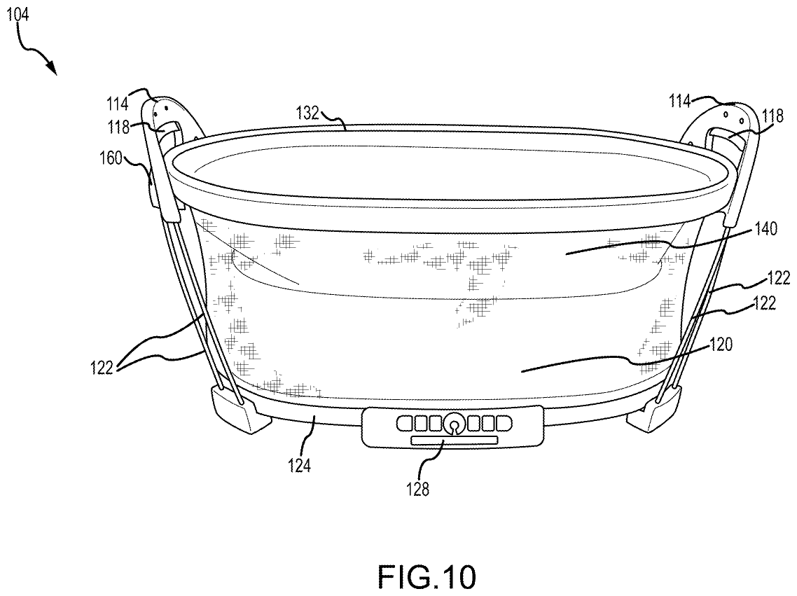

The upper rim 132 of the basket 104 defines an oval shape. The upper rim 132 may be formed in other shapes, such as rectangular, circular, or rectangular with rounded ends or sides. Other shapes for the upper rim 132 are considered to be within the spirit of the disclosure. At the bottom of the basket 104 is a lower edge 124. The lower edge 124 may have a shape or profile similar to the upper rim 132. In some examples the lower edge 124 may have a different shape than the upper rim 132. The lower edge 124 surrounds and defines a bottom of the basket 104. The bottom of the basket 104 provides a surface that may provide support to an infant or may provide support to materials stored within the basket 104. A control panel 128 may secure to the lower edge 124. The control panel 128 is described in further detail below with respect to FIGS. 9-10.

Supports 122 extend between the upper rim 132 and the lower edge 124 in order to provide a rigid or semi-rigid height of the basket 104. The supports 122 are narrow in profile so as to not block the view of the interior of the basket 104. Extending between the upper rim 132 and the lower edge 124 is a side wall 120. The side wall 120 may be a fabric material which provides support but through which light can pass so as to enable a caretaker to view into the interior of the basket 104 from any angle. The bottom of the basket 104 may be formed of the same material as the side wall 120. The material may be a woven fabric material, a plastic material defining holes therein, a rubber material defining holes therein, a natural fiber material, or other such material defining a plurality of holes or openings to provide clear viewing access into the interior of basket 104. In some embodiments, the material may be a mesh panel that is formed of an easy-to-clean rubber, plastic, or other such material. Although shown as a continuous wall, it will be appreciated that in some cases the wall may be formed in various sections so long as appropriate visibility is provided.

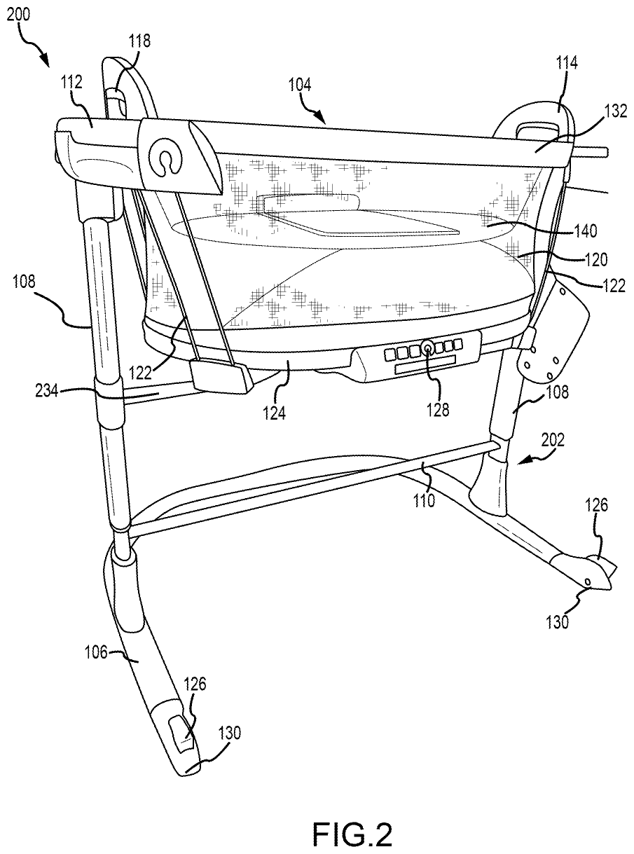

FIG. 2 depicts a perspective view of a bassinet 200 having a crossbar for height adjustment actuation, according to at least some embodiments. The bassinet 200 may have substantially the same structure as the bassinet 100 of FIG. 1, but includes a second crossbar 234 including a latch for actuating the height adjustment of the supports 108. The bassinet 200 includes a frame 202 as well as a basket 104. The bassinet 200 enables the basket 104 to be removed from the frame 202. Additionally, as described above, the basket 104 may rock or pivot with respect to the frame 202, such as to provide a swaying motion for an infant lying therein. The bassinet 200 may be height-adjustable and also provides storage solutions and visibility of an infant for a caregiver as described below. Additionally, the wheels 130 are shown with locking members 126 to prevent rotation of the wheels when engaged by providing a frictional interface against the wheels 130.

The supports 108 each include two lengthwise segments, with a first one coupled to the “U”-shaped foot 106 and a second one of the lengthwise segments coupled to the upper rail 112. The two lengthwise segments of the supports 108 are shown nested, with one contained inside the other concentrically such that the supports 108 extend by the two lengthwise segments moving axially with respect to one another. Locking pins or a locking mechanism may secure the height of the supports 108. The locking pins or mechanism may be released through actuation of a single latch or button on the second crossbar 234. Thus, a user may actuate the latch on the second crossbar 234 with one hand and raise or lower the height of the frame 102 with a second hand. In some embodiments, the supports may include spring elements to assist in raising or lowering the height of supports 108 such that it is a gentle motion that can be accomplished with a single hand. The crossbar 110 and the second crossbar 234 ensure that the height of the frame 202 is equal at each support 108 by ensuring that corresponding segments of the supports 108 move or adjust the same distance with respect to one another. In addition, the crossbar 110 and the second crossbar 234 provide strength against twisting of the frame 202.

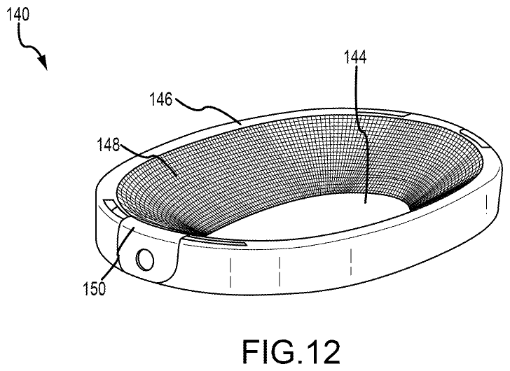

The basket 104 shown in FIG. 2, includes an infant insert 140. Additional details of the infant insert 140 are provided below with respect to FIG. 12. The infant insert 140 couples to the upper rim 132 of the basket 104 such that the insert 140 is suspended above the bottom of the basket 104. In this way, the infant insert 140 maintains a resting surface for an infant nearer the upper rim 132 than the bottom of the basket 104. The infant insert 140 may be formed of the same material as the side wall 120 as well as the bottom of the basket 104. The infant insert 140 may also include padding or cushioning on which the infant rests when placed on the infant insert 140. The distance between the bottom of the insert 140 and the bottom of the basket may be between two and eight inches. The distance between the bottom of the insert 140 and the bottom of the basket 104 positions a young infant at a position more readily accessible to a caregiver. The bottom of the insert 140 is therefore raised towards an upper portion of the basket 104, positioning an infant on a firm surface at a position easy for a caregiver to view and remove or lay the infant down for rest. The sleep surface of the insert 140 is smaller than the sleep surface of the basket 104 and therefor reduces caregiver's need to use bumpers, reducers, or other items to create barriers or reduce the area for sleeping in the bassinet.

The insert 140 may vary in depth, width, and length to provide different configurations and arrangements when coupled to the basket 104. For example, different inserts 140 may be inserted that have different depths, for example with a first, shallower depth intended for use with younger infants and a second, deeper depth for use with somewhat older infants or infants of different sizes, or based on a preference of the caregiver. The different inserts may have sleep surfaces of varying size as well as varying shapes and dimensional ratios. For instance, the sleep surface is smaller than a sleep surface of the basket 104 and a first insert may have a smaller sleep surface than a second insert. The first insert may also position the sleep surface at a different height relative to the upper rim 132 of the basket 104 than the second insert. Various combinations of sizes, shapes, and heights of sleep surfaces and inserts may provide for numerous combinations useful for different purposes or based on caregiver preference. In some examples the insert 140 may be adjustable, for example with an adjustable depth by altering a height of the walls of the insert 140 with a releasable attachment. The sleep surface may also be configurable, for example to fold out into a larger surface area from a first configuration to a second configuration. In the first configuration the sleep surface may have a different shape or size than the second configuration.

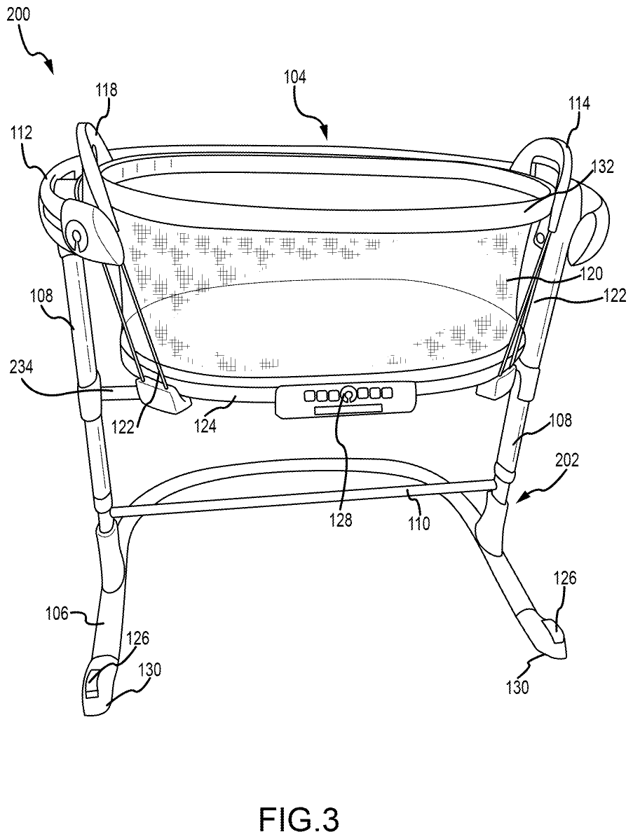

FIG. 3 depicts a perspective view of the bassinet 200 of FIG. 2 with the infant insert 140 removed, according to at least some embodiments. The elements of FIG. 3 include the elements of FIG. 2 as shown and described above. The infant insert 140 is removed in the depiction shown in FIG. 3. The infant insert 140 is secured to the basket 104 with a zipper around the upper rim 132 of the basket 104. A first portion of the zipper may be included with the infant insert 140 around the perimeter of the infant insert. A second portion of the zipper is included around the upper rim 132. The first portion of the zipper and the second portion of the zipper are releasably coupleable together to removably secure the infant insert 140 to the basket 104. In some examples, the zipper around the perimeter may be replaced with other releasable connections such as drawstrings, elastic, buttons, snaps, and the like.

FIG. 4 depicts a perspective view of the frame 102 of the bassinet 100 of FIG. 1, according to at least some embodiments. The frame 102 has a “U”-shaped foot 106 to which a pair of wheels 130 are connected. The wheels 130 are connected to the ends of the “U”-shaped foot 106 such that the bassinet 100 rests on a supporting surface on the “U”-shaped foot 106 as well as the wheels 130. The wheels 130 may be locked with locking members 126 to prevent movement of the bassinet 100 while in use. Though only two wheels 130 are shown in FIG. 4, in some examples there may be fewer or greater numbers of wheels 130, such as wheels arranged around different positions of the “U”-shaped foot 106.

Extending upwards from the “U”-shaped foot 106 are two supports 108. The supports 108 extend perpendicular or substantially perpendicular to the supporting surface or a plane of the “U”-shaped foot 106. In some examples, the supports 108 may extend from one edge, such as a center portion, of the “U”-shaped foot 106 and be angled or cantilevered to maintain the basket in a central position of the bassinet. The bassinet 100 is shown with two supports 108, however, other numbers of supports 108 could be used, such as, one or more than two supports 108. For example, there may be one, three, four, or more supports 108 extending from the “U”-shaped foot 106. The supports 108 may be height-adjustable, either manually or automatically. One example of a manual height adjustment includes a feature depressed by a user (such as a latch) which releases a securing pin and allows the supports 108 to be extended or shortened before re-securing the pin by releasing the latch and allowing the pin to pass through a port in the wall of supports 108. In such examples, the supports 108 may include two or more segments arranged axially and concentrically with respect to each other such that when moved relative to each other along the axis the support 108 is extended or shortened. In an example of an automatic height adjustment, each support 108 may be outfitted with one or more linear actuators. The linear actuators may advance segments or sections of the supports 108 to extend or shorten the length of the supports. In this example, the linear actuators may be controlled through a computing system and activated by a switch depressed by a caregiver.

As shown in FIG. 4, pin holes 142 are provided which may represent a plurality of heights of the supports 108 representing different positions each support 108 can be placed in. For example, there may be a plurality of holes spaced about one inch apart that provide height adjustment by one inch increments to change the height of the upper rail 112 within a range of twenty to thirty-two inches. In some examples, the height of the upper rail 112 may be set by changing the length of the supports 108. The height of the upper rail 112 may also be less than or greater than the range provided above in some embodiments.

The supports 108 are joined by a crossbar 110 which provides strength, rigidity, and support between the supports 108. The crossbar 110 may be a simple beam that extends between the supports 108. The crossbar 110 may also include a height adjustment latch as described below to release the supports 108 for extension or shortening. The height adjustment latch may actuate pins into or out of the pin holes 142 to enable adjustment of the length of the supports. The height adjustment latch may include rigid members in tension extending from pins at the supports 108 to the center of the crossbar 110.

At the upper end of the supports 108, an upper rail 112 provides a connection to the basket 104. The upper rail 112 is shown having a “C”-shape, though other configurations may be used. For example, the upper rail 112 may be formed of multiple segments, such as an upper rail 112 at each of the basket 104. At opposite ends of the upper rail 112 there may be basket connections 116. The basket connections 116 enable the basket 104 to be removed from the frame 102. The basket connections 116 enable rocking of the basket 104 around an axis which passes through both of the basket connections 116. The basket connections may include a first cylindrical element having a first diameter and a second cylindrical element having a second diameter. The second diameter may be larger than the first diameter. The first cylindrical element may couple between the upper rail 112 and the second cylindrical element. Corresponding connection features on the basket 104 may rest on and couple to the second cylindrical element to provide the rotational motion described above. In some examples, the basket connection 116 may include a pivoting member that may act as a bearing to provide rotation to the basket 104.

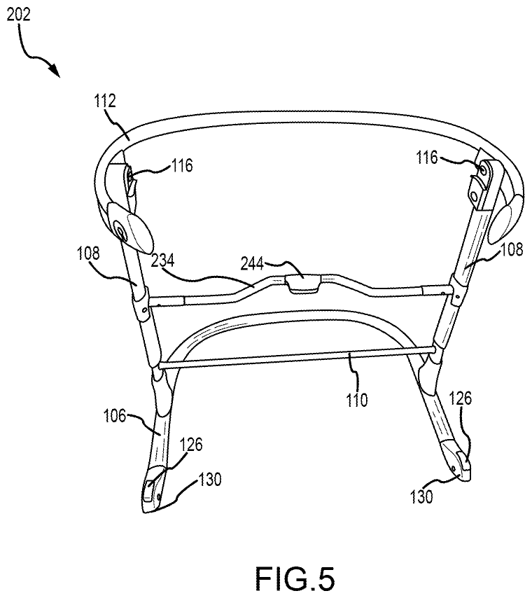

FIG. 5 depicts a perspective view of the frame 202 of the bassinet 200 of FIG. 2, according to at least some embodiments. The frame 202 has the same base configuration described above with the wheels 130 and “U”-shaped foot 106. The upper rail 112 and connected components including the basket connections 116 may be the same as described above.

The supports 108 are joined by a crossbar 110 which provides strength, rigidity, and support between the supports 108. The crossbar 110 may be a simple beam that extends between the supports 108. A second crossbar 234 spans between the supports 108 in the same manner as crossbar 110. The second crossbar 234 includes a latch 244 to actuate the pins or other release mechanism described above to enable adjustment of the height of the supports 108. The latch 244 latch may actuate pins into or out of the pin holes described above to enable adjustment of the length of the supports 108. The latch 244 may include rigid members in tension extending from pins at the supports 108 to the center of the second crossbar 234.

FIG. 6 depicts a perspective view of the bassinet 200 of FIG. 2, according to at least some embodiments. The bassinet 200 includes the same elements as shown and described above. The bassinet 200 is shown with the infant insert 140 secured to the basket 104. The infant insert 140 couples to the edge of the basket 104 around the perimeter of the upper edge. In some examples it may couple with a releasable attachment such as Velcro®, hook and loop fasteners, buttons, snaps, zip closures, or other such releasable attachments. The infant insert 140 may be formed of a single piece or may include multiple pieces, such as a padded section 144 surrounded by perimeter 146 that connects to the basket 104 around the perimeter. The infant insert 140 may be formed of the same material as the side wall 120 and have holes, pores, or openings to provide airflow and visual lines of sight into the infant insert 140.

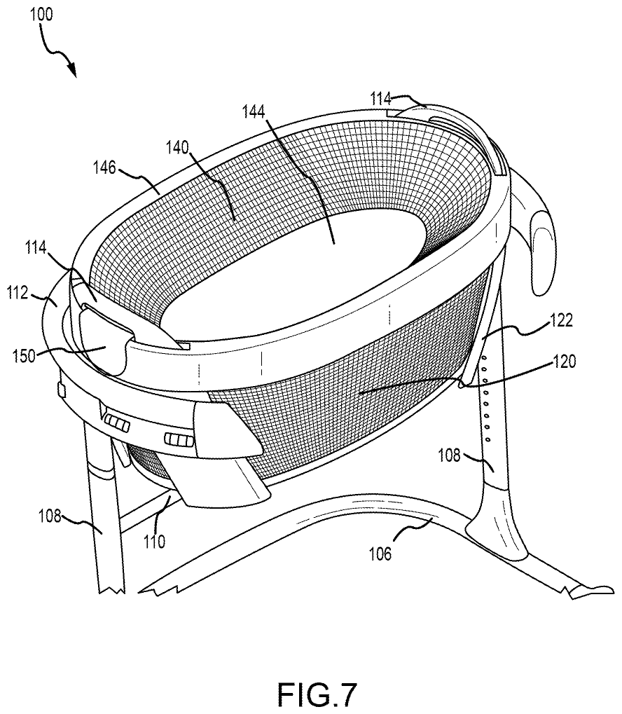

FIG. 7 depicts a basket of a bassinet 100 including an infant insert 140 connected to a frame 102, according to at least some embodiments. The supports 122 are shown as wider beams than in previous figures, but may be exchanged with the narrow support rods shown above. The infant insert 140 couples to the edge of the basket 104 around the perimeter of the upper edge. In some examples it may couple with a releasable attachment such as Velcro®, hook and loop fasteners, buttons, snaps, zip closures, or other such releasable attachments. A tab 150 of the infant insert 140 may extend through an opening defined by the handle 114 to cover a releasable attachment of the infant insert 140. The infant insert 140 may be formed of a single piece or may include multiple pieces, such as a padded section 144 surrounded by perimeter 146 that connects to the basket 104 around the perimeter. The infant insert 140 may be formed of the same material as the side wall 120 and have holes, pores, or openings to provide airflow and visual lines of sight into the infant insert 140. The infant insert 140 provides a surface nearer the upper portion of the basket 104 than the bottom of the basket 104 for an infant to rest on. The infant insert 140 is particularly useful for newborn infants who are unable to roll and can readily be removed as infants grow and age.

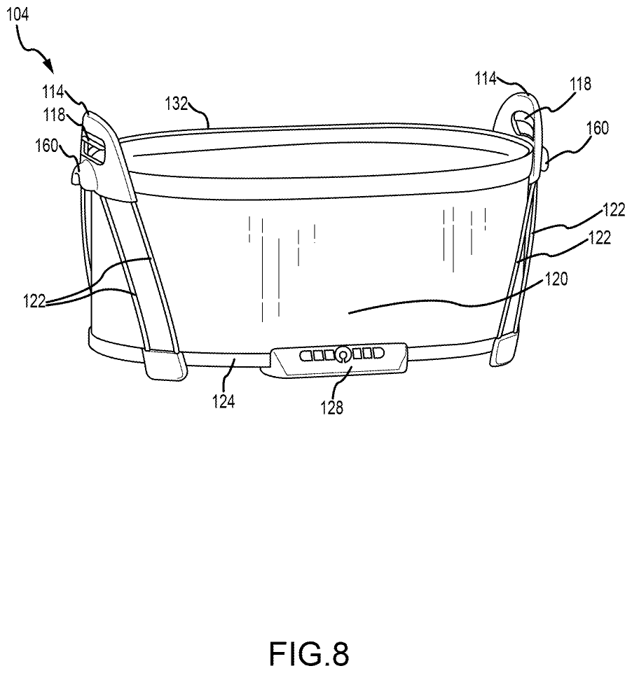

FIGS. 8-9 depict the basket of FIG. 7 removed from the frame 102 with a control panel 128 attached, according to at least some embodiments. The basket 104 includes handles 114 at opposite ends of the basket 104 with release latches 118 inside of each handle 114. The handles 114 may enable a caregiver to lift or carry the basket 104. The release latches 118 actuate a latching mechanism 160 that connects to the basket connections 116 to secure the basket 104 to the frame 102. The latching mechanism 160 still allows the rotation described above, even when the basket 104 is latched to the frame 102.

The latching mechanism 160 is shaped to receive the first cylindrical section of the basket connection 116 within a housing. The housing has a shape that mates with the first cylindrical section and covers or encloses a portion of the first cylindrical section when connected. The housing of the latching mechanism 160 also includes a rotation limit. The rotation limit limits the rotation of the basket 104 relative to the frame 102, by bottoming out or interfering with portions of the basket connection 116. The latching mechanism 160 also includes a portion that mates with the second cylindrical section. When the release latch 118 is actuated, retaining members may release or enable the first cylindrical element to escape from the housing of the latching mechanism 160. This may include pivoting a latch, adjusting a pinching retainer, or other such means to secure the basket connection 116 into the mechanism 160.

The upper rim 132 of the basket 104 defines an oval shape. The upper rim 132 may be formed in other shapes, such as rectangular, circular, or rectangular with rounded ends or sides. Other shapes for the upper rim 132 are considered to be within the spirit of the disclosure. At the bottom of the basket 104 is a lower edge 124. The lower edge 124 may have a shape or profile similar to the upper rim 132. In some examples the lower edge 124 may have a different shape than the upper rim 132. The lower edge 124 surrounds and defines a bottom of the basket 104. The bottom of the basket 104 provides a surface that may provide support to an infant or may provide support to materials stored within the basket 104. A control panel 128 may secure to the lower edge 124. The control panel 128 is described in further detail below with respect to FIGS. 9-10.

Supports 122 extend between the upper rim 132 and the lower edge 124. to provide a rigid or semi-rigid height of the basket 104. The supports 122 are narrow in profile so as to not block the view of the interior of the basket 104. Extending between the upper rim 132 and the lower edge 124 is a side wall 120. The side wall 120 may be a fabric material which provides support but through which light can pass so as to enable a caretaker to view into the interior of the basket 104 from any angle. The bottom of the basket 104 may be formed of the same material as the side wall 120. The material may be a woven fabric material, a plastic material defining holes therein, a rubber material defining holes therein, a natural fiber material, or other such material defining a plurality of holes or openings to provide clear viewing access into the interior of basket 104.

FIG. 10 depict a basket 104 including an infant insert 140 of the bassinet 200 of FIG. 2 resting on a supporting surface, according to at least some embodiments. The basket 104 includes the elements described above and is depicted resting on a supporting surface without a stand, such as the height adjustable frame 202. The infant insert 140 is shown coupled to the basket 104 as described above. The infant insert 140 and basket 104 allows ventilation and clear lines of sight into the resting area of the basket 104.

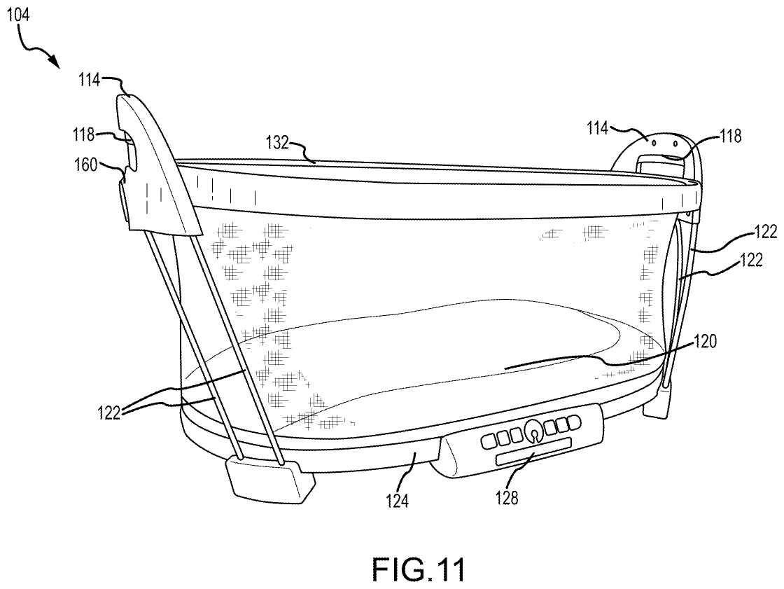

FIG. 11 depicts the basket 104 of the bassinet 200 of FIG. 2 resting on a supporting surface, according to at least some embodiments. The basket 104 is the same basket 104 described above but is depicted with the infant insert 140 removed. The basket 104 provides clear lines of sight into the resting area as well as ventilation.

FIG. 12 depicts an infant insert 140 for connecting to a basket 104 of a bassinet, according to at least some embodiments. The infant insert 140 may be formed of the same material as the side wall 120 described above including various mesh materials, cloth, fabric, rubber, silicone, plastic, natural fiber, and other such articles which provide ventilation and visibility into the bassinet 100. The infant insert 140 includes a padded section 144 where an infant may be laid by a caregiver. The padded section 144 may include additional layers of padding or reinforcement to support a flat surface when an infant is laid thereon. For example, the padded section 144 may include a sandwiched reinforcement layer between the outermost fabric on the top and bottom side of the padded section 144. Around the perimeter 146, the infant insert 140 may have the same shape as the upper rim 132 of the basket 104 such that it fits on the basket securely. As described above, the infant insert may secure to the basket 104 with snaps, buttons, hook and loop fasteners, zippered fasteners or other such releasable fasteners. At either end of the infant insert 140 is a tab 150 that interfaces with the handle 114 of the basket as well as optionally interfacing with the basket connection 116 and/or the latching mechanism 160. Spanning between the padded section 144 and the perimeter 146, an angled sling portion 148 supports the padded section and couples it to the perimeter 146. The angled sling portion 148 may have a tapered shape that transitions from the size and shape of the perimeter 146 and of the upper rim 132 to the shape of the padded section 144. The angled sling portion 148 may suspend below the upper rim 132 when connected to the basket 104. This may form a cradle or cradle portion to support and maintain an infant in the center of the infant insert 140.

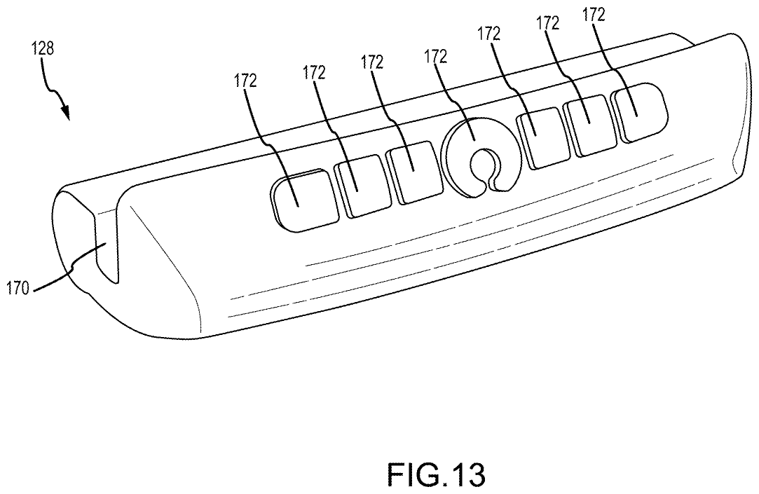

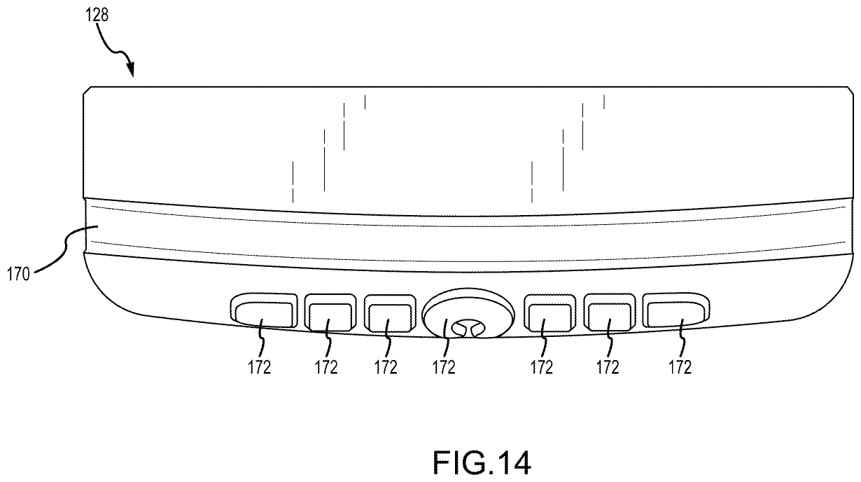

FIGS. 13-14 depict a control panel 128 for bassinet 100, according to at least some embodiments. The control panel 128 may control various aspects of the bassinet 100 including lights, sounds, motions, vibrations, timers, and other such features. The control panel may couple to the basket 104 at the lower edge 124 by a friction fit of the channel 170 over the lower edge 124 or by another type of locking mechanism. The channel 170 may be curved, straight, or have a profile that matches the curvature or profile of the lower edge 124 of basket 104. The control panel 128 may be removable for simplicity and ease of use (for example, to change batteries), as well as enabling placement at any location around the perimeter of the lower edge 124. Control panel 128 includes one or more computer controller or other electronics that are programmed to control various electronic features of bassinet 100. In some examples, the control panel 128 may control vibration, light, or other such settings for predetermined periods of time, such as, for example, 15, 30, or 45 minutes. Control panel 128 may also include settings for intensity, brightness, and other such settings to configure the different operation modes of the control panel 128.

The control panel 128 includes buttons 172 that control various features or actions. In some examples the frame 102 or portions of the basket may be outfitted with lights pointed toward the supporting surface to allow a caregiver to see when in a dark room without providing light to wake a sleeping infant. Such light may be positioned on the bottom of the control panel 128, on the “C”-shaped upper rail 112, or other locations on the frame 102 or basket 104. The control panel 128 may control a vibration or motion setting by providing vibration to the basket 104 or other motion to the basket 104. For example, in some embodiments, the basket connection 116 may include a motorized rocker mechanism that may be controlled by control panel 128 to provide consistent rocking motion to the basket 104. The control panel 128 may also be outfitted with audio or other components to play sounds, produce white noise or music, or perform other soothing tasks for an infant within the basket 104.

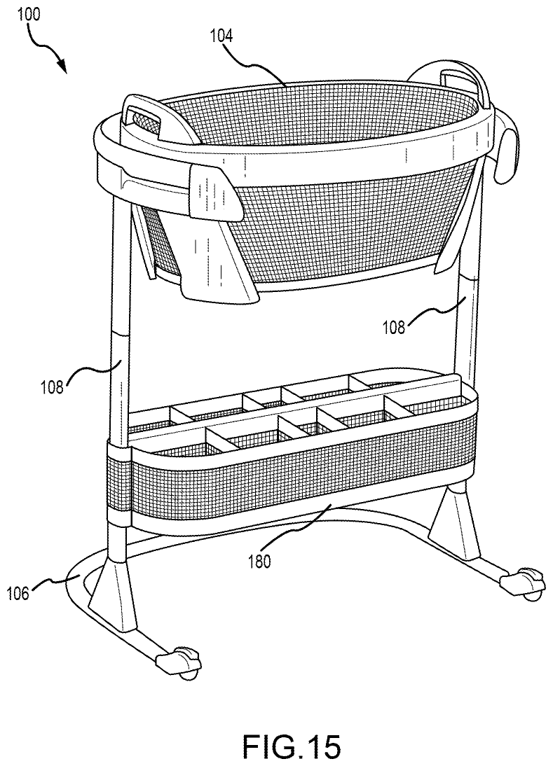

FIG. 15 depicts a bassinet 100 having a storage caddy 180 coupled to the frame 102, according to at least some embodiments. Caddy 180 provides storage in addition to that underneath the infant insert 140. The storage caddy 180 may couple to the supports 108 as well as the crossbar 110. The storage caddy may have compartments of various sizes to receive items such as bottles, wipes, diapers, clothing, bibs, blankets, cloths, toys, and other such baby items. The storage caddy may, in some embodiments, be coupled to only the supports 108 or to only the crossbar 110. In any event, the storage caddy may include an opening or passage to enable access to a latch, such as latch 244 of FIG. 5 to actuate the height change of the bassinet 100. The storage caddy may be formed of any suitable material including those described above, in particular woven, plastic, natural materials.

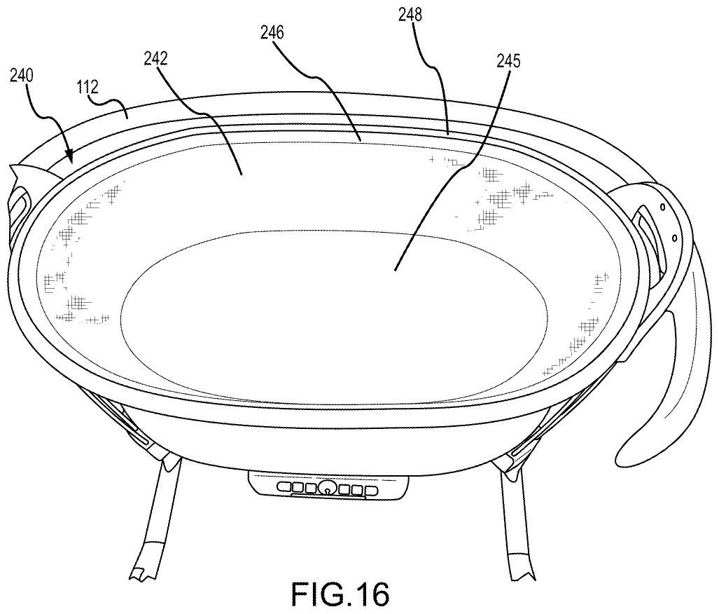

FIG. 16 depicts a top view of a bassinet including a basket, which may be the same as the basket 104 situated within rail 112, with an infant insert 240 connected to the basket. The infant insert 240 includes an infant support portion 245 that has a smaller surface area than the bottom of the basket 104. The reduced surface area may ensure that the infant, when resting in the insert, is unable to roll away. The infant support portion 245 is connected to an upper rim of the basket by a sloped side wall 242 that may be formed of a mesh material, similar to the walls of the basket 104, enabling a line of sight into the infant insert 240 from the surrounding areas. The sloped side wall 242 enables the smaller surface area of the infant support portion 245 and also ensures that infants who are able to roll do not roll away from the center of the basket 104. At the upper edge of the sloped side wall 242 is a first zipper portion 246 coupled to the sloped side wall 242 that interfaces with a second zipper portion 248 coupled to the basket 104. The infant insert 240 may, therefore, be zipped in and out to attach and detach from the basket 104. The zipper connection provides for consistent and constant force on the infant insert around the entire perimeter of the infant insert 240, ensuring that the weight of the infant is evenly distributed and preventing any shifting of the infant insert as the weight of the infant may shift. As described above, other attachment mechanisms and devices may be used that enable releasable connection of the infant insert 240 with the basket 104.

Further embodiments can be envisioned to one of ordinary skill in the art after reading this disclosure. In other embodiments, combinations or sub-combinations of the above disclosed invention can be advantageously made. The example arrangements of components are shown for purposes of illustration and it should be understood that combinations, additions, re-arrangements, and the like are contemplated in alternative embodiments of the present invention. Thus, while the invention has been described with respect to exemplary embodiments, one skilled in the art will recognize that numerous modifications are possible.

The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A bassinet comprising:
a bassinet basket comprising a mesh side panel and a bottom panel, the mesh side panel and bottom panel allowing air movement through the mesh side panel and the bottom panel and allowing visual inspection of an interior of the bassinet basket from a variety of surrounding positions;
an infant insert removably coupleable to an upper portion of the bassinet basket, the infant insert configured to support an infant above the bottom panel of the bassinet basket and forming an open compartment between the bassinet basket and the infant insert; and
a frame having a support portion for resting against a supporting surface and at least one vertical support, and wherein the bassinet basket is operably coupled to the frame such that the bassinet basket is movable in a back and forth rocking motion.

2. The bassinet of claim 1, wherein the infant insert removably couples to the bassinet basket with a zip fastener around the upper portion of the bassinet basket.

3. The bassinet of claim 1, wherein the at least one vertical support is height adjustable.

4. The bassinet of claim 1, wherein the bassinet basket is removable from the frame.

5. The bassinet of claim 1, further comprising a basket coupling portion connected to the frame and comprising two rotational basket couplings that couple to the bassinet basket to allow rotation of the bassinet basket about a single axis passing through the rotational basket couplings and limiting a rotation angle of the bassinet basket about the single axis.

6. The bassinet of claim 5, wherein the two rotational basket couplings are lockable to lock a rotational position of the bassinet basket relative to the frame and prevent further rotation of the bassinet basket about the single axis.

7. The bassinet of claim 1, wherein the bassinet basket comprises a first handle and a second handle at opposite ends of the bassinet basket, and wherein the first handle and the second handle each comprise a removal lock to selectively release or secure the bassinet basket to the frame.

8. The bassinet of claim 1, further comprising a control device coupled to the bassinet basket, the control device configured to selectively at least:
emit light;
impart a vibration to the bassinet basket; or
emit sound.

9. A bassinet comprising:
a basket comprising:
a mesh side panel and a bottom panel, the mesh side panel and a bottom panel allowing air movement through the mesh side panel and the bottom panel and allowing visual inspection of an interior of the basket from a variety of surrounding positions; and
selectively releasable connections for releasably securing the basket to a supporting structure; and
a height-adjustable frame for resting against a supporting surface, the height-adjustable frame comprising:
a height-adjustable vertical support; and
a basket interface portion that releasably engages with the selectively releasable connections to enable selective attachment or removal of the basket from the height-adjustable frame, the basket interface portion enabling limited rotation of the basket about an axis passing through the selectively releasable connections when the basket is secured to the height-adjustable frame.

10. The bassinet of claim 9, further comprising an infant insert releasably securable to an upper portion of the basket, the infant insert comprising an infant support surface positioned above the bottom panel of the basket when secured to the basket, the infant support surface having a smaller surface area than a surface area of the bottom panel.

11. The bassinet of claim 10, wherein the infant insert further comprises a sloped panel connecting the infant support surface and the upper portion of the basket.

12. The bassinet of claim 10, wherein the infant insert couples to the upper portion of the basket with a zippered connection.

13. The bassinet of claim 9, wherein the basket interface portion comprises a selective rotation lock that selectively enables or restricts rotation of the basket about the axis.

14. The bassinet of claim 9, wherein the height-adjustable vertical support and the basket interface portion each comprise a releasable lock that provides a visual and audible indication of locking status.

15. A height-adjustable, rocking bassinet, comprising:
a removable basket comprising:
an upper rim having first connection elements;
a bottom rim;
a bottom panel coupled to the bottom rim;
a mesh side wall coupled to both the upper rim and the bottom rim and defining a side of the removable basket;
a support structure comprising one or more thin supports extending from the upper rim to the bottom rim without substantially impeding visibility from a plurality of surrounding positions into an interior of the removable basket and providing structure for the removable basket; and
a frame comprising:
a height adjustment element enabling vertical positioning of an upper portion of the frame at a plurality of vertical positions; and
second connection elements, wherein the first connection elements and the second connection elements releasably couple together to enable removal of the removable basket from the frame.

16. The height-adjustable, rocking bassinet of claim 15, wherein the first connection elements or the second connection elements comprise a locking feature that selectively locks corresponding first connection elements and second connection elements together to prevent removal of the removable basket until the locking feature is disengaged.

17. The height-adjustable, rocking bassinet of claim 15, wherein the first connection elements and the second connection elements enable rotation of the removable basket about a single axis when the first connection elements and the second connection elements are engaged together.

18. The height-adjustable, rocking bassinet of claim 17, wherein the second connection elements comprise a rotation lock that, when engaged, prevents rotation of the removable basket about the single axis.

19. The height-adjustable, rocking bassinet of claim 15, further comprising an infant insert releasably securable to an upper edge of the removable basket, the infant insert comprising an infant support surface positioned above the bottom panel of the removable basket when secured to the removable basket, the infant support surface having a smaller surface area than a surface area of the bottom panel.

20. The height-adjustable, rocking bassinet of claim 19, wherein the infant insert further comprises a sloped panel connecting the infant support surface and the upper rim.
